(19) United States
(12) Patent Application Publication
(10) Pub. No.: US 2007/0072912 A1
(43) Pub. Date: Mar. 29, 2007

(54) Alicyclic-amine-substituted 4-carboxamido-benzimidazoles as parp-inhibitors and antioxidants

(75) Inventors: Kalman Hideg, Pecs (HU); Tamas Kalai, Cegled (HU); Balazs Sumegi, Pecs (HU)

Correspondence Address: DAVIS WRIGHT TREMAINE LLP, 865 FIGUEROA STREET, SUITE 2400, LOS ANGELES, CA, 90017-2566, US

(73) Assignee: Cedars-Sinai Medical Center, Los Angles, CA (US)

(21) Appl. No.: 10/553,937

(22) PCT Filed: Apr. 27, 2004
(86) PCT No.: PCT/HU04/00043

§ 371 Date: Sep. 21, 2006

(30) Foreign Application Priority Data
Apr. 28, 2003 (HU) P0301154

(51) Int. Cl. A61K 31/4439; A61K 31/4184; C07D 403/02

(52) U.S. Cl. 514/338; 548/305.4; 546/273.4; 514/394

(57) ABSTRACT

Compounds of the formula (I) and their pharmaceutically acceptable or technically applicable acid salts—where in the formula R¹ represents hydrogen, C₍₁₋₄₎alkyl or C₍₁₋₄₎alkoxy R² represents hydrogen, C₍₁₋₄₎alkyl, carboxyl, C₍₁₋₄₎alkoxycarbonyl, carboxamido, aryl or hetero-aryl R³ represents hydrogen, C₍₁₋₄₎alkyl, aryl-methylene, or aryl, Y is a valency bond, a straight or branched chain C₍₁₋₄₎alkene, a carbonyl-amino-C₍₁₋₄₎alkene, or a —S—(CH₂)ₘ— group, where all alkene groups above may be spaced by an arylene group, n represents zero or the integer 1 m represents the integer 1, 2 or 3 Q represents hydrogen, hydroxyl or the oxygen radical (0) or together with the N atom of the adjacent ring forms a +N=O (oxoimmonium) group Z represents a single or double bond and their pharmaceutically acceptable or technically useful salts, processes for their preparation and their biological use as PARP inhibitors and antioxidants.

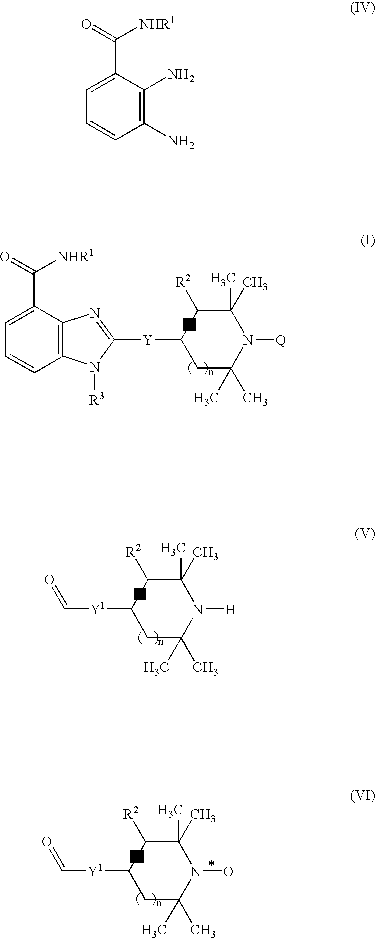

The invention relates to new biologically active chemical compounds, methods for their preparation, pharmaceutical compositions containing the same and methods for their use. More particularly the objects of the invention are 2-sterically hindered alicyclic-amine-substituted 4-carboxamido-benzimidazoles, their salts, their synthesis, their use as new PARP-inhibitors and antioxidants, as well as compositions comprising the new compounds for direct medical use and the use of the new compounds as intermediates for further useful chemicals and their preparation. The new compounds comprise two different bioactive functions—a sterically hindered pyrroli(di)ne or piperidine and a 4-substituted-benzimidazole ring; as a consequence they show both PARP-inhibiting and antioxidant activities.

Abbreviations used in this specification:

PARP=poly(ADP-ribose)polymerase=poly-adenyl-ribosylase

NAD=nicotinamide adenine nucleotide

TBAR=thiobarbituric acid reacting substances

ROS=Reactive Oxidative Species

RNS=Reactive Nitrogen Species

PARP-inhibitors=compounds inhibiting PARP.

The first objects of the present invention are compounds of the general formula (I)— where in the formula
R¹ represents hydrogen, C₍₁₋₄₎alkyl or C₍₁₋₄₎alkoxy
R² represents hydrogen, C₍₁₋₄₎alkyl, carboxyl, C₍₁₋₄₎alkoxycarbonyl, carboxamido, aryl or hetero-aryl
R³ represents hydrogen, C₍₁₋₄₎alkyl, aryl-methylene, or aryl
Y is a valency bond, a straight or branched chain C₍₁₋₄₎alkene, a carbonyl-amino-C₍₁₋₄₎alkene, or a —S—(CH₂)ₘ-group,
where all alkene groups above may be spaced by an arylene group,
n represents zero or the integer 1
m represents the integer 1, 2 or 3
Q represents hydrogen, hydroxyl or the oxygen radical (O.) or together with the N atom of the adjacent ring forms a +N=O (oxoimmonium) group
Z represents a single or double bond
   and their pharmaceutically acceptable or technically useful salts. Compounds of formula (I) include molecules of general formula (I)¹—where in the formula R1, R2, R3, Z and n represent the same as above while
Y¹ is a valency bond, straight or branched C₍₁₋₄₎alkene, or carbonyl-amino-C₍₁₋₄₎alkene
   where the alkene group in all of the above groups may be spaced by an arylene group.

In this specification the meaning of the above substituents in the general formulae is always the same and they are therefore not repeated herein.

The new compounds of the invention can be used per se as the basis for pharmaceutical media especially as protective agents against several forms of diseases caused by Reactive Oxidative Species (ROS) and Reactive Nitrogen Species (RNS) or diseases which are based on PARP activation or both. They can also be used as intermediates in the chemical production of medically effective materials in the same field.

It is known that the final cause of cell damage in the case of vascular diseases is the oxidative stress of the endothelial cells and of the blood cells (thrombocytes and red blood cells). Oxidative stress causes lipid peroxidation, and this destroys the structure of the lipid bilayer of plasma membrane, which damages ion transport proteins. In ischemic neurodegenerative damages Ca²⁺ overload, ROS and RNS are the main contributors. The ROS, e.g. H₂O₂ induces both sodium and calcium influx into the cells. In the presence of iron, the oxidizing agent hydrogen peroxide produces lipid peroxidation and at the same time increases the intracellular calcium concentration. Thus, in the presence of hydrogen peroxide, parallel measurements of lipid peroxidation and concentration of intracellular free calcium ion are appropriate methods for the determination of oxidative cell destruction. [Detection of lipid peroxidation is possible by way of methods using thiobarbituric acid reacting substances (TBAR). Intracellular free calcium ion can be determined by using a fluorescent intracellular calcium indicator.]

It is also known that PARP is a nuclear protein that is a critical component of the cellular response to DNA damage. There is considerable evidence suggesting that PARP inhibitors can play an important role in repair of DNA damage. Several PARP inhibitors were therefore synthetized and have shown efficacies in several animal disease models of cancer, ischemia and inflammation. Various 2-substituted-4-carboxamido-benzimidazoles, mono- and bicyclic carboxamides, bi-, tri- and tetracyclic lactames and some other heterocyclic molecules were proposed as PARP-inhibitors (J. Med. Chem. 2003. 46. 210-213; Review: Idrugs 2001.4 (7):804-812). However, none of them contain alicyclic stable nitroxide or its amine precursor functions.

We experienced earlier that certain sterically hindered amines e.g. 2,2,5,5-tetramethyl-2,5-dihydro-1H-pyrrole-3-carboxylic acid [3-(1,3-dioxo-1,3-dihydro-isoindol-2-yl)-propyl]-amide with antiarrhythmic activity metabolized to the corresponding non-toxic nitroxide: 1-hydroxy-2,2,5,5-tetramethyl-2,5-dihydro-1H-pyrrole-3-carboxylic acid [3-(1,3-dioxo-1,3-dihydro-isoindol-2-yl)-propyl]-amide (J. Med. Chem., 1986, 29, 1138-1152., Free Rad. Biol. & Med., 1997, 22, 909-916). Both compounds exhibit reduction of the oxidative damages caused by reactive oxidative intermediates formed during reperfusion.

It was also demonstrated before that when certain other antiarrhythmic drugs, e.g. mexiletine, tocainide were modified with a sterically hindered alicyclic nitroxide or its precursor amine the molecules not just preserved or even enhanced their antiarrhythmic activity but gained a strong antioxidant effect by which they turned capable of an in situ scavenging in statu nascendi of those highly reactive ROS and RNS which are responsible for oxidative damages (J. Pharmacol. Exp. Ther. 2000, 292, 838-845; J. Pharmacol. Exp. Ther. 2000, 295, 563-571). It is also known that a great variety of sterically hindered 5- and 6-membered nitroxides and their amine precursors protect against damages caused by H₂O₂ and radiation (J. Med. Chem. 1998, 41, 3477-3492).

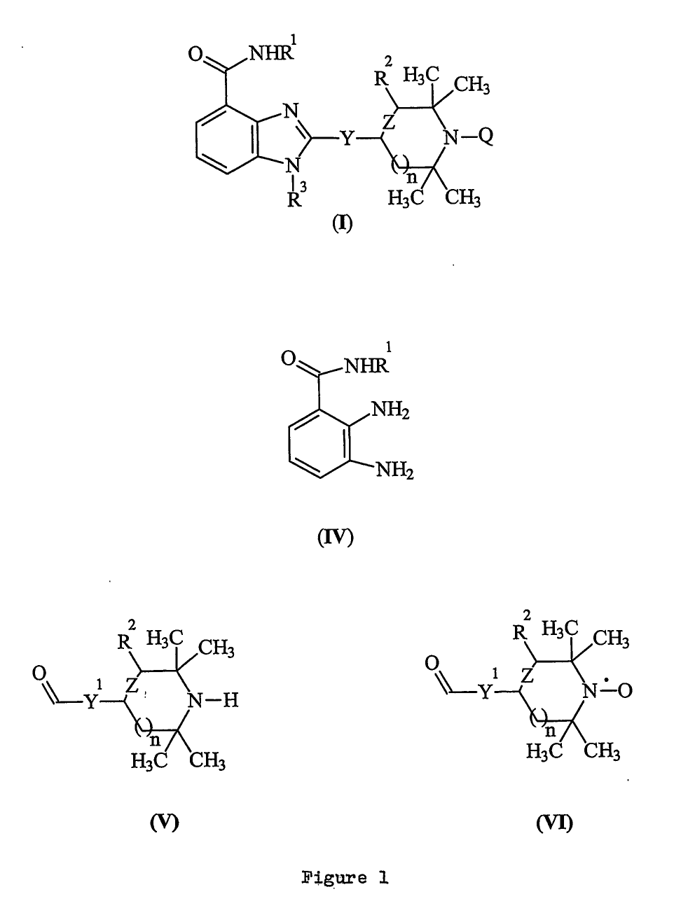

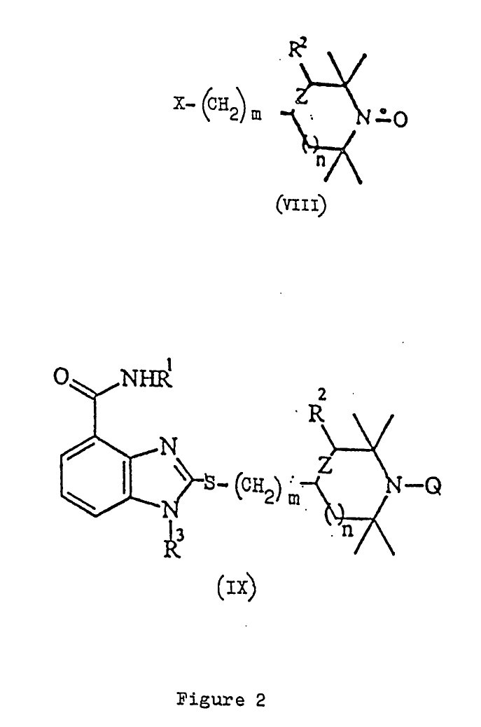

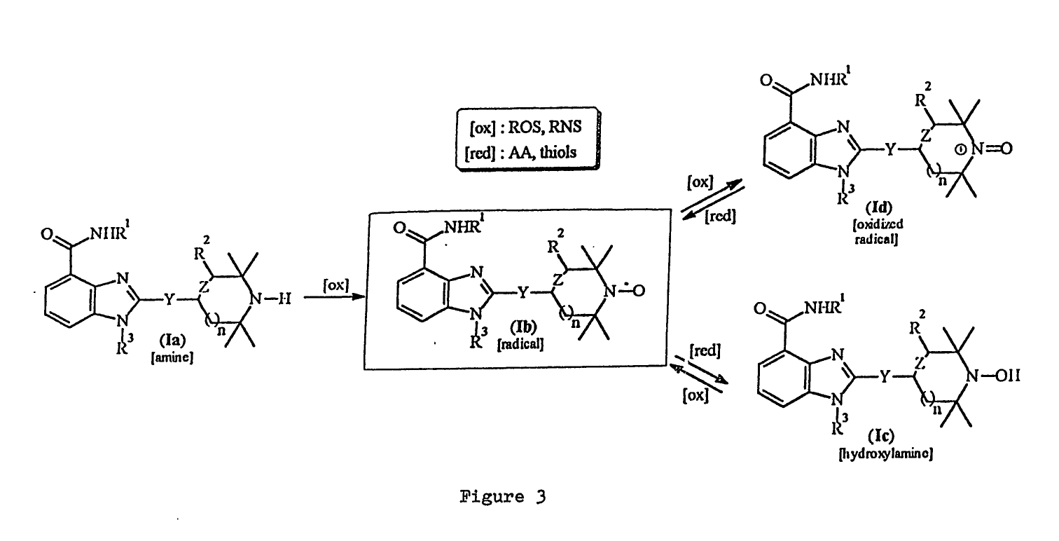

The basis of the present invention was the recognition that properly designed sterically hindered amines and their oxidized derivatives are capable to fulfill similar antioxidant function as e.g. do sterically hindered phenols, indoles, sulphides and disulphides. This is shown by the reaction scheme in FIG. 3.

The sterically hindered amines and non-toxic radicals may offer the exceptional advantage that they can fulfill the function of multi-step protectors in an antioxidant cascade system. The sterically hindered pyrroli(di)ne or piperidine-N-oxyl derivatives comprised in the compounds of general formula (I) and their amine precursors of general formula (Ia) according to the present invention exhibit a protective effect against damages caused by H₂O₂ and other reactive oxygen species; they also exhibit a cardioprotective effect. In addition the presence of a 4-carboxamido-benzimidazole-group in the same molecule makes these compounds capable to inhibit the damages of DNA via inhibition of the PARP activity.

Thus it is another basis of the present invention that the new molecules containing both of these functions exhibit both a high PARP-inhibiting activity and a capability for scavenging damages caused by toxic ROS and RNS events.

The new compounds of the general formula (I) according to the invention are capable to exist in the form of general formula (Ia)-(Ib)-(Ic)-(Id) (see FIG. 3). Compounds of general formula (Ia) according to the invention metabolize in the organism to the corresponding nitroxides of general formula (Ib) which equilibrate to diamagnetic N-hydroxyl compounds of general formula (Ic) or can be oxidized further up to oxoimmonium compounds of general formula (Id). The N-hydroxyl is able to be oxidized back to nitroxide.

All forms of the compounds of the general formula (I) namely amines of the general formula (Ia), nitroxides of the general formula (Ib), N-hydroxyl compounds of the general formula (Ic) and the oxoimmonium compounds of the general formula (Id) and salts of these compounds are subject of the present invention.

Both the amines and the N-hydroxyl compounds are water-soluble in their salt form, formed with pharmaceutically acceptable mineral acids or organic acids. Such salts are the hydrochlorides, hydrobromides, sulphates, phosphates, phosphites, borates, lactates, ascorbates, acetates, fumarates, formiates, oxalates, tosylates, tartarates, maleates, citrates, gluconates, besylates etc. The salts represent subjects of the present invention. However in addition to the above salts other salts with mineral or organic acids may be of technological use on the course of preparation of the products. Such salts include e.g. the oxalates. Also the technologically useful salts are subjects of the present invention.

The combination of two different types of biologically active molecules according to the invention results in a scavenger-type drug with functions of antioxidants in cascade of defence combined with PARP inhibiting effects. This is verified in the biological examples presented concerning compounds of the general formula (I) according to the invention.

Thus first objects of the present invention are new compounds of the general formula (I) and their pharmaceutically acceptable salts.

Compounds according to the invention may contain along with the substituted benzimidazole a piperidine, pyrrole or a pyrrolidine ring as the heterocyclic ring. These may be tetramethyl-substituted and may contain further substituents such as trifluoro-methyl-phenyl-, hydroxy-, acetyl-, alkoxy-groups. The compounds contain benzimidazole-carboxamide groups which can be primary acid amides or secondary acid-(alkylated) amides.

Preferred compounds are those where the substituents contain C₍₁₋₄₎alkyl as alkyl, C₁₋₄alkoxy as alkoxy, C₁₋₄alkoxycarbonyl as alkoxycarbonyl, phenyl as aryl, piperidine, pyrrole or pyrrolidine groups as heteroaryl groups, a C₁₋₄alkene as alkene, 6 or 12 membered arylene such as phenylene as arylene groups in any of the substituents where such groups are mentioned. Compounds of preference specifically include the following molecules which are readily synthetized and show advantageous biological properties in their free base form, in the form of their pharmaceutically acceptable salts or other forms according to the invention:
2-(1-oxyl-2,2,5,5-tetramethyl-2,5-dihydro-1H-pyrrol-3-yl)-1H-benzimidazole 4-carboxylic acid amide radical
2-(2,2,5,5-tetramethyl-2,5-dihydro-1H-pyrrol-3-yl)-1H-benzimidazole 4-carboxylic acid amide
4-(4-carbamoyl-1H-benzimidazol-2-yl)-1-oxyl-2,2,5,5-tetramethyl-pyrrolidine 3-carboxylic acid methyl ester radical
4-(4-carbamoyl-1H-benzimidazol-2-yl)-2,2,5,5-tetramethyl-pyrrolidine-3-carboxylic acid methyl ester 2-(4-bromo-1-oxyl-2, 2, 5, 5-tetramethyl-2,5-dihydro-1H-pyrrol-3-yl)-1H-benzimidazole 4-carboxylic acid amide radical
2-(4-bromo-2,2,5,5-tetramethyl-2,5-dihydro-1H-pyrrol-3-yl)-1H-benzimidazole 4-carboxylic acid amide
2-(1-oxyl-4-phenyl-2,2,5,5-tetramethyl-2,5-dihydro-1H-pyrrol-3-yl)-1H-benzimidazole 4-carboxylic acid amide radical
2-(4-phenyl-2,2,5,5-tetramethyl-2,5-dihydro-1H-pyrrol-3-yl)-1H-benzimidazole 4-carboxylic acid amide
2-[1-oxyl-2,2,5,5-tetramethyl-4-(3-trifluoromethyl-phenyl)-2,5-dihydro-1H-pyrrol-3-yl]-1H-benzimidazole 4-carboxylic acid amide radical
2-[2,2,5,5-tetramethyl-4-(3-trifluoromethyl-phenyl)-2,5-dihydro-1H-pyrrol-3-yl]-1H-benzimidazole 4-carboxylic acid amide
2-[4-(1-oxyl-2,2,5,5-tetramethyl-2,5-dihydro-1H-pyrrol-3-yl)-phenyl]-1H-benzimidazole 4-carboxylic acid amide radical
2-[4-(2,2,5,5-tetramethyl-2,5-dihydro-1H-pyrrol-3-yl)-phenyl]-1H-benzimidazole 4-carboxylic acid amide
2-(1,2,2,5,5-pentamethyl-2,5-dihydro-1H-pyrrol-3-yl)-1H-benzimidazole 4-carboxylic acid amide
2-(1-acetyl-2,2,5,5-tetramethyl-2,5-dihydro-1H-pyrrol-3-yl)-1H-benzimidazole 4-carboxylic acid amide
2-(1-methoxy-2,2,5,5-tetramethyl-2,5-dihydro-1H-pyrrol-3-yl)-1H-benzimidazole 4-carboxylic acid amide
2-[4-(dibenzofuran-4-yl)-1-oxyl-2,2,5,5-tetramethyl-2,5-dihydro-1H-pyrrol-3-yl)-phenyl]-1H-benzimidazole 4-carboxylic acid amide radical
2-[4-(dibenzofuran-4-yl)-2,2,5,5-tetramethyl-2,5-dihydro-1H-pyrrol-3-yl)-phenyl]-1H-benzimidazole 4-carboxylic acid amide
(1-hydroxy-2,2,6,6-tetramethyl-1,2,3,6-tetrahydro-pyridin-4-yl)-1H-benzimidazole 4-carboxylic acid amide
2-(2,2,6,6-tetramethyl-1,2,3,6-tetrahydro-pyridin-4-yl)-1H-benzimidazole 4-carboxylic acid amide
2-[4-(1-oxyl-2,2,5,5-tetramethyl-2,5-dihydro-1H-pyrrol-3-yl-methoxy)-phenyl]-1H-benzimidazole 4-carboxylic acid amide radical
2-[4-(2,2,5,5-tetramethyl-2,5-dihydro-1H-pyrrol-3-yl-methoxy)-phenyl]-1H-benzimidazole 4-carboxylic acid amide
2-[3-methoxy-4-(1-oxyl-2,2,5,5-tetramethyl-2,5-dihydro-1H-pyrrol-3-yl-methoxy)-phenyl]-1H-benzimidazole 4-carboxylic acid amide radical
2-[3-methoxy-4-(2,2,5,5-tetramethyl-2,5-dihydro-1H-pyrrol-3-yl-methoxy)-phenyl]-1H-benzimidazole 4-carboxylic acid amide
2-(5-oxyl-4,4,6,6-tetramethyl-4,6-dihydro-5H-thieno[2,3-c]pyrrol-2-yl)-1H-benzimidazole 4-carboxylic acid amide radical
2-(4,4,6,6-tetramethyl-4,6-dihydro-5H-thieno[2,3-c]pyrrol-2-yl)-1H-benzimidazole 4-carboxylic acid amide
2-(1-oxyl-2,2,5,5-tetramethyl-2,5-dihydro-1H-pyrrol-3-yl)-1H-benzimidazole 4-carboxylic acid isopropylamide radical
2-(2,2,5,5-tetramethyl-2,5-dihydro-1H-pyrrol-3-yl)-1H-benzimidazole 4-carboxylic acid isopropylamide
1-(2,2,5,5-tetramethyl-2,5-dihydro-1H-pyrrol-3-yl-methyl)-1H-benzimidazole 4-carboxylic acid amide radical;
1-(2,2,6,6-tetramethyl-1,2,3,6-tetrahydro-pyridin-4-yl)-1H-benzimidazole 4-carboxylic acid amide.
2-(1-oxyl-2,2,5,5-tetramethyl-2,5-dihydro-1H-pyrrol-3-yl-methylsulphanyl)-1H-benzimidazole 4-carboxylic acid amide radical
2-(2,2,5,5-tetramethyl-2,5-dihydro-1H-pyrrol-3-yl-methyl-sulphanyl)-1H-benzimidazole 4-carboxylic acid amide
2-(1-oxyl-2,2,6,6-tetramethyl-1,2,3,6-tetrahydro-pirydin-4-yl-methylsulphanyl)-1H-benzimidazole 4-carboxylic acid amide
2-(2,2,6,6-tetramethyl-1,2,3,6-tetrahydro-pyridin-4-yl-methylsulphanyl)-1H-benzimidazole 4-carboxylic acid amide and its hydrochloric acid salt.

A further object of the present invention are processes to obtain the compounds according to the general formula (I). The processes to be used differ depending on the substituents.

Processes for the preparation of compounds of the general formula (I¹)—where R¹, R², Y¹, Z and n have the meaning as stated above—include reactions of suitably substituted carboxamides of the general formula (IV)—where R¹ has the meaning as stated above—with heterocyclic derivatives of the general formulae (V) or (VI)—where R², Y¹, Z and n have the meaning as stated above.

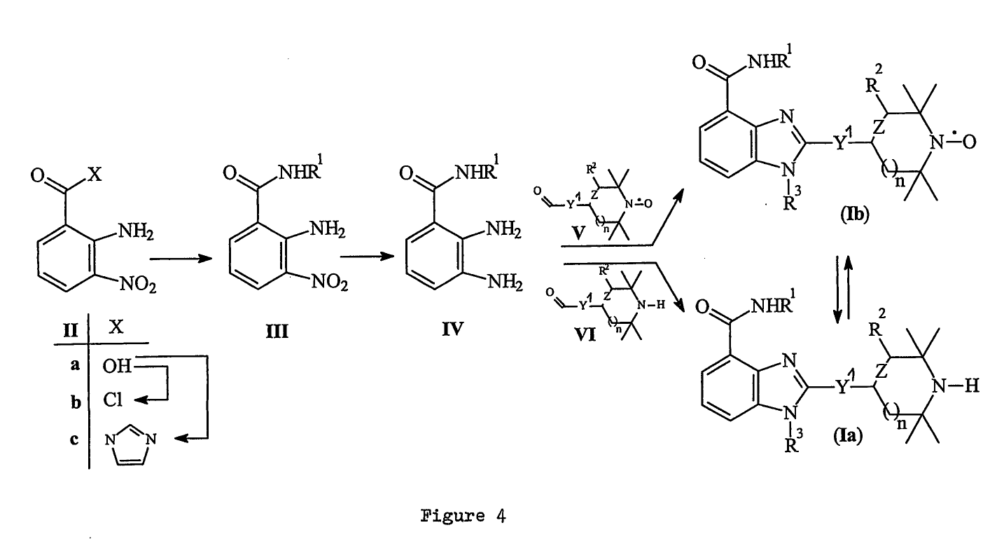

The carboxamides of general formula (IV) which are used as starting materials are known or can be prepared by known methods. One method is shown in the reaction scheme seen in FIG. 4. The same reaction scheme also shows the synthesis of the compounds of the general formula (I).

The condensation of carboxamides of the general formula (IV) with the heterocyclic molecules of general formula (V) or (VI) lead to the benzimidazole ring closure while also ensuring the suitable substitution of the benzimidazole ring. This reaction can be accomplished in the presence of a suitable organic solvent such as toluene, benzene, chloroform etc. The optimal solvent depends also on the substituents of the benzimidazole ring. The reaction takes place normally under gentle heating at 20 to 80° C. Isolation and purification of the products can be usually achieved by known methods.

The compound of the formula VII is obtained from the compound of the formula IV by way of a base catalysed reaction under reflux with carbon disulphide in the presence of an organic solvent such as THF, DMF, DMSO, or alcohols such as ethanol, methanol, dichloromethane or others. Suitable catalysts are e.g. NaOMe, NaOH, DBU, K₂CO₃ etc.

Processes for the preparation of another group of compounds of general formula (I)—namely compounds of general formula (IX)—where R¹, R², Z, Q, n and m have the meaning as stated above—by way of reacting a compound of the general formula VII—where R¹ has the meaning as above—with an alkylating agent of general formula VIII—where R², Z, Q, n and m have the same meaning as stated above and
X stands for a leaving group capable to react with the mercapto group to form a thioether
   and optionally changing the substituents Q by way of oxydation and/or reduction to obtain the desired change in the substituents Q.

Preferred reagents are the correspondingly substituted alkyl-halogenides or alkyl-sulphonates such as members of the group selected of the type alkyl chloride, alkyl bromide, alkyl-iodide, alkyl-mesylate, alkyl-tosylate, alkyl-triflate (Synthesis 1980, 914-916: Can. J. Chem. 1985, 63, 940-943) in the presence of an a equivalent amount of a suitable base. Suitable bases for this purpose are e.g. triethyl amine, K₂CO₃, KOH. Alkylation can be accomplished at a temperature of about 30 to 80° C. in an appropriate solvent such as e.g. THF, DMF, DMSO, acetone, ethanol or methanol etc.

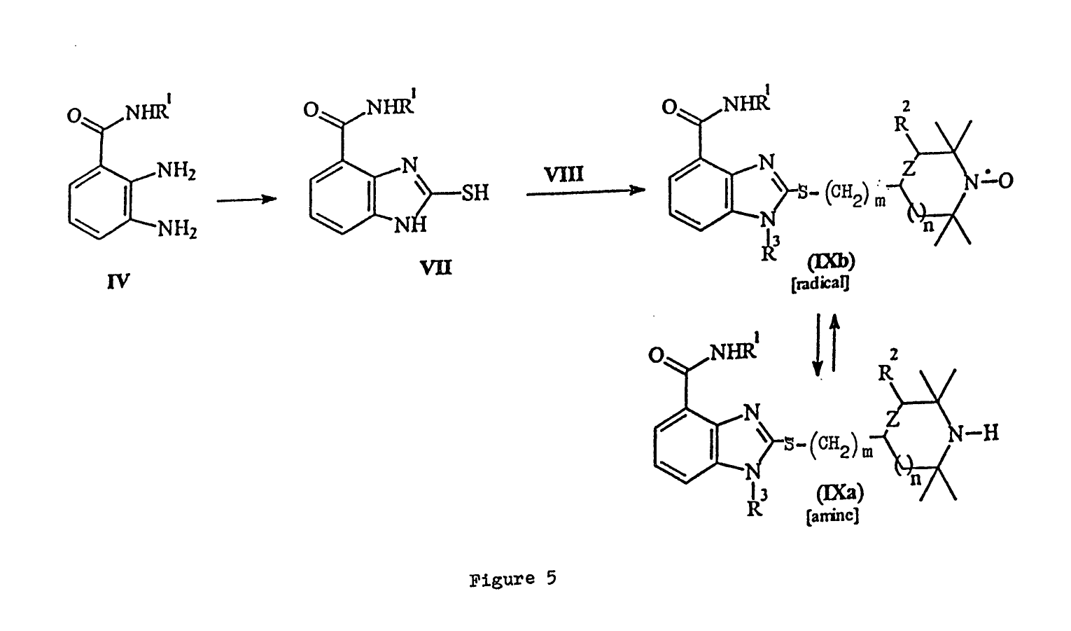

In the S-alkylation reaction a compound of the general formula IXb is formed first (see reaction scheme on FIG. 5)) and this can be transferred into the IXa, IXc and IXd forms in the same manner as the other compounds of the general formula (I) as seen on reaction scheme FIGS. 3 and 4 respectively.

Some compounds of the general formula (I) are sparingly soluble in water and some are water-soluble. They form pharmaceutically acceptable or technically useful water-soluble salts with acids as already indicated above. Purification can be accomplished by way of salt-formation.

The nitroxides of general formula (Ib) [including compounds of general formula (IXb)] can be transformed into compounds of formula (Ia) [including compounds of general formula (IXa)] by way of reduction. Reduction may be accomplished by reacting with pulverised iron under gentle heating in concentrated acetic acid.

The products can be isolated by diluting the reaction mixture, making it alkaline and extracting the active ingredient e.g. with a halogenated solvent such as chloroform. The free base of Ia can be transferred into its salt by addition of acids.

The nitroxides of general formula (Ib) can be transferred into the N-hydroxylamine form of general formula (Ic) by way of heating in ethanol in the presence of an acid. This product can be precipitated from the reaction mixture by addition of a suitable solvent where the product is less soluble.

The N-hydroxylamines can be oxidized into the N-oxides of the general formula (Id) using gentle oxidizing methods.

All products can be purified using chromatography or re-crystallization.

A further object of the present invention are pharmaceutical compositions comprising as an active ingredient compounds of the general formula (I) or their pharmaceutically acceptable salts. The present invention includes formulations comprising compounds of the general formula (I) in either of their possible forms (Ia), (Ib), (Ic) and (Id) including the compounds (I¹a), (I¹b), (I¹c), (I¹d), (IXa), (IXb). The drugs can be administered orally in solid or liquid forms, transdermally, in different injectable forms or infusions, or any other form such as sublingual, pernasal, rectal. The pharmaceutical formulations are prepared and formulated accordingly.

Yet other objects of the present invention are methods of treatment of patients in need of such treatment where there is need for scavanging damages caused by ROS or RNS events or of PARP-inhibition or both by way of administering an effective amount of a compound of the general formula (I) in an adequate dosage form containing the effective dose. Typical of such damages are for example the following diseases which can be treated or prohibited by way of administration of effective amounts of compounds of the general formula (I) or their salts: coronary diseases, ischemia, inflammation. They may be used to enhance killing of tumour cells on the course of radiotherapy or chemotherapy.

The doses which can be used for the above purpose vary to a high degree depending on the intended use and the molecule and its substituents employed.

Yet another object of the present invention is the process to produce the pharmaceutical compositions comprising as active ingredient a compound of the general formula (I) in either of their possible forms (Ia), (Ib), (Ic) and (Id) to obtain formulations which can be administered for scavenging damages caused by ROS or RNS events or of PARP-inhibition or both. The formulation for oral, injectable, parenteral, rectal, transdermal or other uses into tablets, pellets, solutions, injectables, patches etc. can be achieved principally in the known manner with usual pharmaceutical additives which do not modify the stability and activity of the active ingredients in a manner which is not advantageous.

Details of the invention are disclosed in the following examples without the intention of limitation.

### I. CHEMICAL EXAMPLES

First general methods of synthesis of the molecules are described followed by tables with the data related to compounds synthesized.

General methods for preparing compounds of general formula (I) are illustrated in the reaction scheme seen on FIG. 4. The meaning of the substituents is the same as indicated above in the specification.

### Example I.1

### Synthesis of 2-amino-3-nitrobenzamide of General Formula (III)

A suspension of 2-amino-3-nitrobenzoic acid (1.82 g, 10.0 moles) heated under reflux for 3 hours in thionyl chloride (10 mL) and the thionyl chloride is removed by vacuum distillation. The residual solid is suspended in THF (20 mL) and 25% aqueous ammonia solution (20 mL) is added in portions with stirring within 15 min. The mixture is allowed to stay over-night, the orange precipitate is filtered and air-dried to give 2-amino-3-nitrobenzamide (900 mg, 49%); mp 238-239° C.; ν max (cm⁻¹) 3420, 3180, 1680, 1580, 1555.

### Example I.2

A mixture of 2-amino-3-nitrobenzoic acid (1.82 g, 10.0 mmoles) and 1,1-carbonyldiimidazole (1.62 g 10.0 mmoles) is refluxed for 30 min in dry THF (40 mL) and a 25% aqueous ammonia solution (20 mL) or the corresponding primary or secondary amine is added in one portion with stirring. The mixture is allowed to stay overnight, the orange precipitate is filtered and air-dried to give 2-amino-3-nitrobenzamide (1.52 g, 84%) or 2-amino-3-nitro-(N-substituted)-benzamide.

### Example I.3

Synthesis of 2,3-diamino-benzamides of the General Formula (IV)

Pd/C (200 mg) is added to a stirred mixture of 2-amino-3-nitrobenzamide (1.81 g, 10.0 mmoles) or 2-amino-3-nitro-(N-substituted)-benzamide and ammonium-formiate (3.78 g, 0.06 mol) in methanol (40 mL) or some other appropriate solvent and the mixture is stirred at 40° C. for 2 hours. The mixture is then filtered through Cellite, washed with methanol (40 mL) or the used solvent, evaporated and the residue is purified by way of crystallyzation or flash column chromatography to give 2,3-diaminobenzamide as a pale brown light sensitive solid (800 mg, 53%), mp 103-105° C.; ν max (cm⁻¹) 3330, 3170, 1630, 1600, MS m/z (%): 151 (M⁺, 70), 134 (72), 106 (100) 79 (38).

The same method can be used for 2,3-diamino-(N-substituted) benzamides.

### Example I.4

### Synthesis of 2-Substituted 4-carboxamido-benzimidazoles (Method A)

A mixture of 2,3-diamino-benzamide (1,51 g, 10.0 mmoles) or a 2,3-diamino-(N-substituted)-benzamide (10.0 mmoles) and a suitable paramagnetic aldehyde (of general formula V) or diamagnetic aldehyde (of general formula VI) (10.0 mol), and toluene-p-sulphonic acid monohydrate (95 mg, 0.5 mmoles) is refluxed in toluene (40 mL) or in an other appropriate solvent till all the starting compounds are consumed (4-6 hours) under Dean and Stark apparatus. Then the solvent is evaporated in vacuo, the residue dissolved in CHCl₃ (50 mL) or in some other halogenated solvent, and an appropriate oxidant such as activated MnO₂ (4.30 g, 50.0 mmoles) is added and the mixture is stirred and refluxed for about 6 hours. The mixture was filtered through Cellite, evaporated and the residue was purified by flash column chromatography (CHCl₃/Et₂O or CHCl₃/MeOH) or crystallization to give compound Ia or Ib (yield: 39-73%).

The aldehydes used are known see e.g.: Hideg et al. Synthesis (1980) 911-914; (1991) 616-620; Csekö et al. Can. J. Chem. (1985) 63 940-943; Sár, P. C. et al. Synth. Comm., (1995) 25, 2929-2940; Kálai et al. Synthesis (1998) 1476-1482; (1999) 973-980; Hankovszky et al. Synthesis (1980) 914-916.

### Example I.5

General method for reducing nitroxide radicals of the general formula Ia) (Q=O.) to diamagnetic alicyclic secondary amines of the general formula (Ib) (Q=H) (Method B).

Upon addition of iron powder (224 mg, 4.0 mmoles) to a stirred solution of the paramagnetic compound of general formula (Ia) (2.0 mmoles) in acetic acid (7 ml) and gentle heating (max. 60° C.) for 30 min., the reaction mixture is diluted with water (20 mL) and filtrated. The filtrate is basified with solid potassium carbonate, extracted with chloroform (2×20 ml), dried and evaporated. The residue is purified by flash chromatography (CHCl₃/MeOH) or acidified with ethanol saturated with hydrochloride gas. The white crystalline hydrochloride salt of the product of the general formula (Ib) is precipitated from EtOH/Et₂O solution (yield: 48-65%).

### Example I.6

Method for reducing nitroxide radicals of general formula (Ia) (Q=O.) to diamagnetic alicyclic N-hydroxylamines of general formula (Ic) (Q=OH) (Method C):

A solution of the paramagnetic compound of general formula (Ia) (1.0 mmoles) in ethanol saturated with hydrochloride gas (10 ml) is refluxed for 1 hour, then diluted with diethyl ether to precipitate the diamagnetic hydroxylamine HCl salt of the general formula (Ic). The basic compound is obtained in white solid from EtOH/Et₂O solution (yield: 53-64%).

### Example I.7

### 4-Carboxamido-1-(2,2,5,5-tetramethyl-2,5-dihydro-1H-pyrrol-3-ylmethyl)-benzimidazole

The mixture of 3-carboxamido-benzimidazole (805 mg, 5,0 mmoles), 3-bromo-methyl-2,2,5,5-tetramethyl-2,5-dihydro-1H-pyrrole (1.16 g, 5.0) mmoles and potassium hydroxide (280 mg, 5.0 mmoles) is refluxed in methanol or other suitable solvent (25 mL) for about 3 hours. The inorganic salt is filtered off, the filtrate evaporated and the residue purified by flash column chromatography (CHCl₃/Et₂O) to give 970 mg (62%) of the title compound.

### Example I.8

### Preparation of 2-mercapto-4-carboxamido-benzimidazole-(VII)

To the solution of 2,3-cyanamido-benzamide (1,51 g, 10.0 mmoles) and carbon-disulfide (760 mg, 10,0 mmoles) in THF (20 mL) the solution of 1.0 Mole sodium methylate (0.5 mL) in methanol is added. After refluxing for an hour the reaction mixture is left alone overnight. The precipitated crystals are filtered and washed with ether (10 mL). 900 mg (46%) of the title product are obtained. Mp.: 354-356 C (decomp.) MS m/z (%): 193 (M⁺, 90), 176(100), 148(33), 105(20), 90(33).

### Example I.9

### Preparation of 2-(1-oxy-2,2,5,5-tetramethyl-2,5-dihydro-1H-pirrol-3-yl-methylsulphanyl)-1H-benzimidazole 4-carboxamide radical (Ib)

The compound 2-mercapto-4-carboxamido-benzimidazole (1,93 g, 10,0 mmoles) and potassium hydroxide (560 mg, 10,0 mmoles) are dissolved in 25 ml of methanol (or some other suitable solvent) and the alkylating agent of general formula (VIII) (10,0 mmoles) is added. The solution is refluxed for about 2 hours. When cool, the inorganic salt is filtered off, the solvent is evaporated and the residue is purified by chromatographic means (using chloroform/ether or chloroform/methanol). 1,55 g of the title product are obtained (45%).

In accordance with the above general methods a series of compounds of general formula (I) was prepared. The compounds with their formulae, characteristics as well as PARP inhibiting and antioxydative effects are shown in Table I.

### II. BIOLOGICAL ACTIVITY STUDIES

### Example II.1

Assay to test inhibitory effects of benzimidazole derivatives on PARP enzyme in vitro.

Poly-ADP-ribose polymerase was isolated from rat liver based on a known method (Anal Biochem 1995, 227, 1-13; 2000, 59, 937-945). The potential inhibitory effect of benzimidazole derivatives were tested in this assay system. The PARP activity was determined in 130 μl reaction mixture contained 100 mM Tris-HCl buffer, pH 8.0, 10 mM MgCl₂, 10% glycerol, 1.5 mM DTT, 1 mM [Adenine-2,8-³H] NAD⁺ (4.500 cpm/nmol), 10 μg activated DNA and 10 μg histones. The incubation time was 15 minutes, and the reaction was stopped by addition of trichloro-acetic acid (8%). After addition of 0.5 mg albumine, precipitation was allowed to proceed for at least 20 minutes on ice, and the insoluble material was collected on a glass filter washed with 5% perchloric acid. The protein-bound radio-activity was determined by a LS-200 Beckman scintillation counter. Data shown in Table I are IC₅₀ values in nM.

### Example II.2

Protecting effect of benzimidazole derivatives against H₂O₂ induced cell death determined in WRL-68 human liver cell line. (Antiox 1, % of protection comparing to control values):

Cell culture. WRL-68 human liver cell line was from American Type Culture Collection (Rockville, Md.). Cell lines were grown in humidified 5% CO₂ atmosphere at 37° C. and maintained in culture as mono-layer adherent cells in Dulbecco's Modified Eagle's Medium containing 1% antibiotic-antimycotic solution (Sigma, St. Louis, Mo.) and 10% fetal calf serum. Cells were passaged at intervals of 3 days.

Detection of cell survival. Cells were seeded into 96-well plates at a starting density of 2.5×10⁴ cell/well and cultured overnight in humidified 5% CO₂ atmosphere at 37° C. The following day H₂O₂ was added to the medium at the indicated concentrations either alone or in the presence of 10 μM of the protecting agent (benzimidazole derivatives). Three hours later the medium was removed and 0.5% of the water soluble mitochondrial dye (3-(4,5-dimethylthiazol-2-yl]-2,5-diphenyl-tetrazolium bromide (MTT⁺) was added. Incubation was continued for 3 more hours, the medium was removed and the metabolically reduced water-insoluble blue formasan dye was solubilised by acidic isopropanol. Optical densities were determined by an Anthos Labtech 2010 ELISA reader (Wien, Austria) at 550 nm wave length. All experiments were run in at least 6 parallels and repeated 3 times. Data of Table I are the concentration of benzimidazoles (in nM) at which the rate of H₂O₂-induced cell death was inhibited by 50%.

### Example II.3

Hydroxyl Radical Scavenging of Benzimidazole Derivates (Antiox 2):

Hydroxyl radical formation was detected using the oxidant-sensitive non-fluorescent probe benzoic acid which is hydroxylated to 2, 3 or 4-hydroxy-benzoic acid (J. Biol. Chem. (1996) 271 40-47). Hydroxylation of benzoic acid results in the appearance of intensive fluorescence which makes possible the fluorescence spectroscopic monitoring of the hydroxylation reactions excitation 305 nm emission 407 nm. The reaction was studied in a 2.5 ml reaction volumes containing 20 mM potassium phosphate buffer (pH 6.8) 0.1 mM benzoic acid, 0.1 mM H₂O₂ and 20 μM Fe²⁺-EDTA. Data of Table I show the concentration of benzimidazoles (in nM) at which the rate of hydroxyl radical induced hydroxylation is inhibited by 50%.

Statistical Analysis.

Data were presented as means ±S.E.M. For multiple comparison of groups ANOVA was used. Statistical difference between groups was established by paired or unpaired Student's test, with Bonferroni correction.

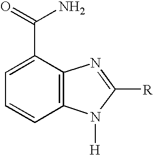

| | | | | | | | | Antiox2 |
|---|---|---|---|---|---|---|---|---|
| | | | | | | PARP | Antiox1 | IC₅₀ |
| Compound | R | Method(Yield) | mp ° C. | m/z (EI) | Formula | IC₅₀ nM | IC₅₀ nM | nM |
| 1 | 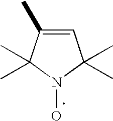 | A(51%) | 248-250 | 299 (M⁺, 19), 269(37), 223(51), 41(100) | C₁₆H₁₉N₄O₂299.35 | 721 | 23.3 | 0.48 |
| 2 | 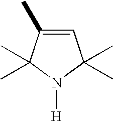 | B(65%) | 239-241 | 284(M⁺, 1), 269(100), 252(91), 224(14) | C₁₆H₂₀N₄O 284.36 | 345 | 92.1 | 2.1 |
| 3 | 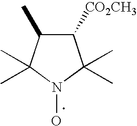 | A(55%) | 255-257 | 359(M⁺, 12), 246(60), 215(62), 41(100) | C₁₈H₂₃N₄O₄359.40 | ND* | 85.9 | 0.9 |
| 4 | 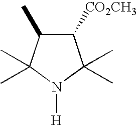 | B(59%) | >260 | 344(M⁺, 1), 312(6), 246(100), 229(54) | C₁₈H₂₄N₄O₃344.41 | 216 | 43.2 | 3.1 |
| 5 | 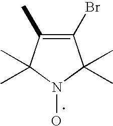 | A(53%) | 142-145 | 377/379(M⁺, 13/13), 363/365(36/36), 268(100), 251(82) | C₁₆H₁₈BrN₄O₂378.24 | 201 | 13.0 | 2.8 |
| 6 | 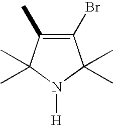 | B(65%) | 249-251 | 362/364(M⁺, <1), 347/349(63/63), 250(48), 42(100) | C₁₆H₁₉BrN₄O 363.25 | 137 | 16.7 | 3.4 |
| 7 | 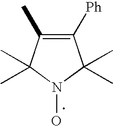 | A(57%) | 257-259 | 375(M⁺, 11), 345(27), 162(93), 145(100) | C₂₂H₂₃N₄O₂375.45 | 1500 | 93.2 | 12.5 |
| 8 | 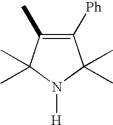 | B(48%) | 256-258 | 360(M⁺, 2), 345(100), 328(40), 285(13) | C₂₂H₂₄N₄O 360.45 | 310 | 98.5 | 0.38 |
| 9 | 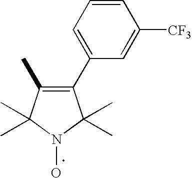 | A(56%) | 253-255 | 443(M⁺, 65), 413(72), 398(82), 381(100) | C₂₃H₂₂F₃N₄O₂443.44 | 149 | 17.3 | 11.2 |
| 10 | 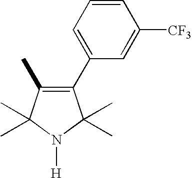 | B(50%) | 224-226 (2 HCl) | 428(M⁺, 2), 413(100), 396(46), 353(11) | C₂₃H₂₃F₃N₄O 428.45 | 133 | 25.5 | 13.4 |
| 11 | 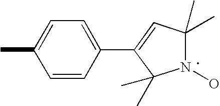 | A(45%) | 254-256 | 375(M⁺, 8), 345(100), 327(22), 237(41) | C₂₂H₂₃N₄O₂375.45 | 78 | 49 | 7.2 |
| 12 | 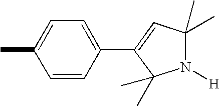 | B(52%) | 149-151 | 360(M⁺, <1), 345(100), 328(24), 313(6) | C₂₂H₂₄N₄O 360.45 | 98 | 33.2 | 4.5 |
| 13 | 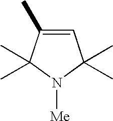 | A(61%) | 173-175 | 298(M⁺, 4), 283(47), 269(98), 252(100) | C₁₇H₂₂N₄O 298.38 | 42 | 42 | 73 |
| 14 | 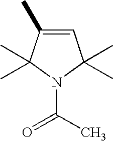 | A(73%) | 139-141 | 326(M⁺, 10), 311(52), 252(62), 43(100) | C₁₈H₂₂N₄O₂326.39 | 49 | 66.8 | 81 |
| 15 | 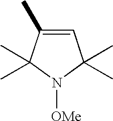 | A(49%) | >260 | 314(M⁺, 11), 299(100), 282(22), 268(18) | C₁₇H₂₂N₄O₂314.38 | 61 | 68.3 | 113 |
| 16 | 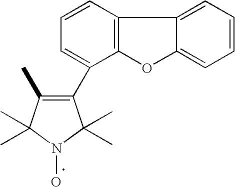 | A(44%) C (64%) | 160-162 195-197 | 465(M⁺, 77), 451(40), 435(100), 420(84) | C₂₈H₂₅N₄O₃465.53 | 1800 | 79.15 | 5.3 |
| 17 | 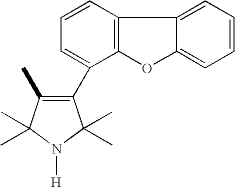 | B(55%) | 223-225 | 450(M⁺, 2), 435(100), 418(26), 375(11) | C₂₈H₂₆N₄O₂450.54 | 8200 | 48.0 | 70 |
| 18 | 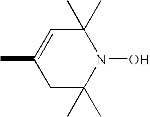 | A, C(53%) | 235-237 | 314(M⁺, 14), 299(100), 283(72), 237(53) | C₁₇H₂₃ClN₄O₂350.85 | 26 | 23 | 9.6 |
| 19 | 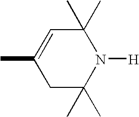 | B(45%) | >260 | 298(M⁺, 23), 283(81), 266(29), 42(100) | C₁₇H₂₂N₄O 298.38 | 14 | 83 | 1.6 |
| 20 | 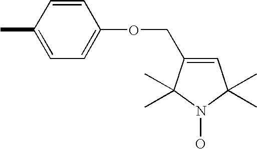 | A(43%) ] C(60%) | 144-146 174-176 | 405(M⁺, 12), 375(19), 108(75), 41(100) | C₂₃H₂₅N₄O₃405.47 | 564 | 40.0 | 13.2 |
| 21 | 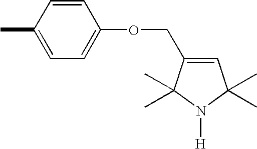 | B(57%) | 198-200 | 390(M⁺, 2), 375(29), 122(65), 108(100) | C₂₃H₂₆N₄O₂390.48 | 572 | 29.2 | 4.3 |
| 22 | 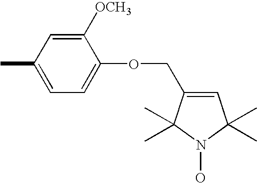 | A(46%) | 244-246 | 435(M⁺, 10), 405 (10), 122(81), 108(100) | C₂₄H₂₇N₄O₄435.50 | 472 | 27.3 | 14.1 |
| 23 | 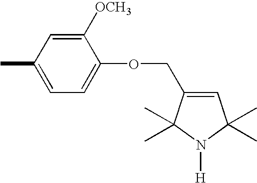 | B(40%) | 234-235 | 420(M⁺, 3), 405(25), 122(71), 108(100) | C₂₄H₂₈N₄O₃420.51 | 432 | 0.4 | 3.8 |
| 24 | 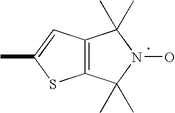 | A(39%) | >260 | 355(M⁺, 10), 341(56), 325(100), 308(34) | C₁₈H₁₉N₄O₂S 355.43 | 3400 | 7.6 | 27 |
| 25 | 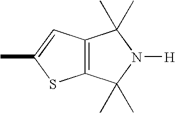 | B(55%) | >260 | 340(M⁺, 6), 325(100), 308(53), 280(10) | C₁₈H₂₀N₄OS 340.44 | 354 | 5.2 | 10.5 |
| 26 | 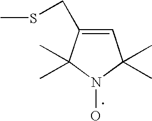 | D(45%) | 249-251 | 345(M⁺, 20), 315(18), 300(13) 193(100), | C₁₇H₂₁N₄O₂S 345.43 | | | |
| 27 | 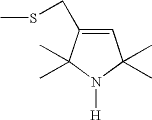 | B(49%) | 209-211 (2 HCl salt) | 330(M⁺, 2), 315(22), 176(10) 122(100), | C₁₇H₂₂N₄OS 330.44 | | | |
| 28 | 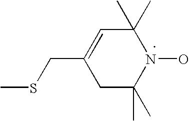 | D(38%) | 102-104 | 359(M⁺, 2), 329(18), 196(42), 41(100) | C₁₈H₂₃N₄O₂S 359.46 | | | |
| 29 | 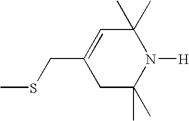 | B(40%) | 245-246 | 344(M⁺, 6), 329(52), 136(75), 55(100) | C₁₈H₂₄N₄OS 344.47 | | | |

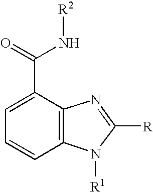

| | | | | | | | | | | Antiox 2 |
|---|---|---|---|---|---|---|---|---|---|---|
| | | | | Method | | | | PARP | | IC₅₀ |
| Compund. | R | R¹ | R² | (Yield) | mp ° C. | m/z (EI) | Formula | IC_{50 nM} | Antiox 1 IC₅₀ nM | nM |
| 30 | 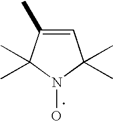 | H | i-Pr | A (46%) | 241-243 | 341 (M⁺, 30), 327 (45), 311 (72), 223 (100) | C₁₉H₂₅N₄O₂341.43 | 10000> | 30 | 2.1 |
| 31 | 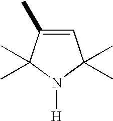 | H | i-Pr | B (51%) | 231-233 | 326 (M⁺, <1), 311 (100), 252 (21), 224 (4) | C₁₉H₂₆N₄O 326.44 | 10000> | 23 | 2.3 |
| 32 | H | 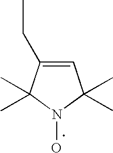 | H | (62%) | 247-249 | 313 (M⁺, 48), 299 (15), 283 (24), 41 (100) | C₁₆H₂₀N₃O 270.35 | 10000> | 32 | 1.8 |

*ND: not determined

1-13. (canceled)

14. A compound of the formula 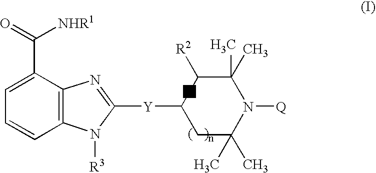
or a pharmaceutically acceptable or technically applicable salt thereof, wherein
R¹ represents hydrogen, C₍₁₋₄₎alkyl, or C₍₁₋₄₎alkoxy;
R₂ represents hydrogen, C₍₁₋₄₎alkyl, carboxyl, C₍₁₋₄₎alkoxycarbonyl, carboxamido, aryl, or hetero-aryl;
R³ represents hydrogen, C₍₁₋₄₎alkyl, aryl-methylene, or aryl;
Y is a valency bond, a straight or branched chain C₍₁₋₄₎alkene, a carbonyl-amino-C₍₁₋₄₎alkene, or a —S—(CH₂)ₘ— group;
n represents zero or the integer 1;
m represents the integer 1, 2, or 3;
Q represents hydrogen, hydroxyl, or the oxygen radical (O.), or together with the N atom of the adjacent ring forms a +N=O (oxoimmonium) group;
Z represents a single or double bond; and
wherein any or all alkene groups may be spaced by an arylene group.

15. The compound of formula (I) or pharmaceutically acceptable or technically applicable salt thereof according to claim 14, wherein
one or more of the aryl substituents are phenyl; the hetero-aryl substituent is piperidine, pyrrole, or pyrrolidine; and/or one or more of the arylene groups are 6 or 12 membered arylene.

16. The compound of formula (I) or pharmaceutically acceptable or technically applicable salt thereof according to claim 14, wherein the compound is selected from the group consisting of
2-(1-oxyl-2,2,5,5-tetramethyl-2,5-dihydro-1H-pyrrol-3-yl)-1H-benzimidazole 4-carboxylic acid amide radical;
2-(2,2,5,5-tetramethyl-2,5-dihydro-1H-pyrrol-3-yl)-1H-benzimidazole 4-carboxylic acid amide;
4-(4-carbamoyl-1H-benzimidazol-2-yl)-1-oxyl-2,2,5,5-tetramethyl-pyrrolidine 3-carboxylic acid methyl ester radical;
4-(4-carbamoyl-1H-benzimidazol-2-yl)-2,2,5,5-tetramethyl-pyrrolidine-3-carboxylic acid methyl ester;
2-(4-bromo-1-oxyl-2,2,5,5-tetramethyl-2,5-dihydro-1H-pyrrol-3-yl)-1H-benzimidazole 4-carboxylic acid amide radical;
2-(4-bromo-2,2,5,5-tetramethyl-2,5-dihydro-1H-pyrrol-3-yl)-1H-benzimidazole 4-carboxylic acid amide;
2-(1-oxyl-4-phenyl-2,2,5,5-tetramethyl-2,5-dihydro-1H pyrrol-3-yl)-1H-benzimidazole 4-carboxylic acid amide radical;
2-(4-phenyl-2,2,5,5-tetramethyl-2,5-dihydro-1H-pyrrol-3-yl)-1H-benzimidazole 4-carboxylic acid amide;
2-[1-oxyl-2,2,5,5-tetramethyl-4-(3-trifluoromethyl-phenyl)-2,5-dihydro-1H-pyrrol-3-yl]-1H-benzimidazole 4-carboxylic acid amide radical;
2-[2,2,5,5-tetramethyl-4-(3-trifluoromethyl-phenyl)-2,5-dihydro-1H-pyrrol-3-yl]-1H-benzimidazole 4-carboxylic acid amide;
2-[4-(1-oxyl-2,2,5,5-tetramethyl-2,5-dihydro-1H-pyrrol-3-yl)-phenyl]-1H-benzimidazole 4-carboxylic acid amide radical;
2-[4-(2,2,5,5-tetramethyl-2,5-dihydro-1H-pyrrol-3-yl)-phenyl]-1H-benzimidazole 4-carboxylic acid amide;
2-(1,2,2,5,5-pentamethyl-2,5-dihydro-1H-pyrrol-3-yl)-1H-benzimidazole 4-carboxylic acid amide;
2-(1-acetyl-2,2,5,5-tetramethyl-2,5-dihydro-1H-pyrrol-3-yl)-1H-benzimidazole 4-carboxylic acid amide;
2-(1-methoxy-2,2,5,5-tetramethyl-2,5-dihydro-1H-pyrrol-3-yl)-1H-benzimidazole 4-carboxylic acid amide;
2-[4-(dibenzofuran-4-yl)-1-oxyl-2,2,5,5-tetramethyl-2,5-dihydro-1H-pyrrol-3-yl)-phenyl]-1H-benzimidazole 4-carboxylic acid amide radical;
2-[4-(dibenzofuran-4-yl)-2,2,5,5-tetramethyl-2,5-dihydro-1H-pyrrol-3-yl)-phenyl]-1H-benzimidazole 4-carboxylic acid amide;
(1-hydroxy-2,2,6,6-tetramethyl-1,2,3,6-tetrahydro-pyridin-4-yl)-1H-benzimidazole 4-carboxylic acid amide;
2-(2,2,6,6-tetramethyl-1,2,3,6-tetrahydro-pyridin-4-yl)-1H-benzimidazole 4-carboxylic acid amide;
2-[4-(1-oxyl-2,2,5,5-tetramethyl-2,5-dihydro-1H-pyrrol-3-yl-methoxy)-phenyl]-1H-benzimidazole 4-carboxylic acid amide radical;
2-[4-(2,2,5,5-tetramethyl-2,5-dihydro-1H-pyrrol-3-yl-methoxy)-phenyl]-1H-benzimidazole 4-carboxylic acid amide;
2-[3-methoxy-4-(1-oxyl-2,2,5,5-tetramethyl-2,5-dihydro-1H-pyrrol-3-yl-methoxy)-phenyl]-1H-benzimidazole 4-carboxylic acid amide radical;
2-[3-methoxy-4-(2,2,5,5-tetramethyl-2,5-dihydro-1H-pyrrol-3-yl-methoxy)-phenyl]-1H-benzimidazole 4-carboxylic acid amide;
2-(5-oxyl-4,4,6,6-tetramethyl-4,6-dihydro-5H-thieno[2,3-c]pyrrol-2-yl)-1H-benzimidazole 4-carboxylic acid amide radical;
2-(4,4,6,6-tetramethyl-4,6-dihydro-5H-thieno[2,3-c]pyrrol-2-yl)-1H-benzimidazole 4-carboxylic acid amide;
2-(1-oxyl-2,2,5,5-tetramethyl-2,5-dihydro-1H-pyrrol-3-yl)-1H-benzimidazole 4-carboxylic acid isopropylamide radical;
2-(2,2,5,5-tetramethyl-2,5-dihydro-1H-pyrrol-3-yl)-1H-benzimidazole 4-carboxylic acid isopropylamide;
1-(2,2,5,5-tetramethyl-2,5-dihydro-1H-pyrrol-3-yl-methyl)1H-benzimidazole 4-carboxylic acid amide radical;
1-(2,2,6,6-tetramethyl-1,2,3,6-tetrahydro-pyridin-4-yl)-1H-benzimidazole 4-carboxylic acid amide;
2-(1-oxyl-2,2,5,5-tetramethyl-2,5-dihydro-1H-pyrrol-3-yl-methylsulphanyl)-1H-benzimidazole 4-carboxylic acid amide radical;
2-(2,2,5,5-tetramethyl-2,5-dihydro-1H-pyrrol-3-yl-methyl-sulphanyl)-1H-benzimidazole 4-carboxylic acid amide;
2-(1-oxyl-2,2,6,6-tetramethyl-1,2,3,6-tetrahydro-pirydin-4-yl-methylsulphanyl)-1H-benzimidazole 4-carboxylic acid amide; and
2-(2,2,6,6-tetramethyl-1,2,3,6-tetrahydro-pyridin-4-yl-methylsulphanyl)-1H-benzimidazole 4-carboxylic acid amide.

17. The compound of formula (I) or pharmaceutically acceptable or technically applicable salt thereof according to claim 14, wherein the salt is formed with inorganic or organic acids.

18. The compound of formula (I) or pharmaceutically acceptable or technically applicable salt thereof according to claim 14, wherein said salt is an oxalate, a hydrochloride, a hydrobromide, a sulphate, a phosphate, a phosphite, a borate, a lactate, an ascorbate, an acetate, a fumarate, a formiate, a tosylate, a tartarate, a maleate, a citrate, a gluconate, or a besylate.

19. A pharmaceutical composition for the treatment of a disease which can be favorably influenced by PARP inhibition and/or scavenging oxidative stress, comprising an effective dose of a compound of the formula 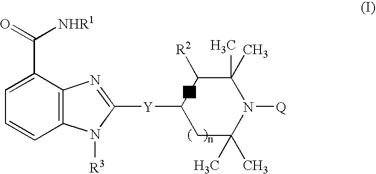
or a pharmaceutically acceptable or technically applicable salt thereof, wherein
R¹ represents hydrogen, C₍₁₋₄₎ alkyl, or C₍₁₋₄₎ alkoxy;
R² represents hydrogen, C₍₁₋₄₎ alkyl, carboxyl, C₍₁₋₄₎ alkoxycarbonyl, carboxamido, aryl, or hetero-aryl;
R³ represents hydrogen, C₍₁₋₄₎ alkyl, aryl-methylene, or aryl;
Y is a valency bond, a straight or branched chain C₍₁₋₄₎alkene, a carbonyl-amino-C₍₁₋₄₎alkene, or a —S—(CH₂)ₘ— group;
n represents zero or the integer 1;
m represents the integer 1, 2, or 3;
Q represents hydrogen, hydroxyl, or the oxygen radical (O.), or together with the N atom of the adjacent ring forms a +N=O (oxoimmonium) group;
Z represents a single or double bond; and
wherein any or all alkene groups may be spaced by an arylene group.

20. The pharmaceutical composition according to claim 19, wherein
one or more of the aryl substituents are phenyl;
the hetero-aryl substituent is piperidine, pyrrole, or pyrrolidine; and/or
one or more of the arylene groups are 6 or 12 membered arylene.

21. The pharmaceutical composition according to claim 19, wherein the compound is selected from the group consisting of
2-(1-oxyl-2,2,5,5-tetramethyl-2,5-dihydro-1H-pyrrol-3-yl)-1H-benzimidazole 4-carboxylic acid amide radical;
2-(2,2,5,5-tetramethyl-2,5-dihydro-1H-pyrrol-3-yl)-1H-benzimidazole 4-carboxylic acid amide;
4-(4-carbamoyl-1H-benzimidazol-2-yl)-1-oxyl-2,2,5,5-tetramethyl-pyrrolidine 3-carboxylic acid methyl ester radical;
4-(4-carbamoyl-1H-benzimidazol-2-yl)-2,2,5,5-tetramethyl-pyrrolidine-3-carboxylic acid methyl ester;
2-(4-bromo-1-oxyl-2,2,5,5-tetramethyl-2,5-dihydro-1H-pyrrol-3-yl)-1H-benzimidazole 4-carboxylic acid amide radical;
2-(4-bromo-2,2,5,5-tetramethyl-2,5-dihydro-1H-pyrrol-3-yl)-1H-benzimidazole 4-carboxylic acid amide;
2-(1-oxyl-4-phenyl-2,2,5,5-tetramethyl-2,5-dihydro-1H pyrrol-3-yl)-1H-benzimidazole 4-carboxylic acid amide radical;
2-(4-phenyl-2,2,5,5-tetramethyl-2,5-dihydro-1H-pyrrol-3-yl)-1H-benzimidazole 4-carboxylic acid amide;
2-[1-oxyl-2,2,5,5-tetramethyl-4-(3-trifluoromethyl-phenyl)-2,5-dihydro-1H-pyrrol-3-yl]-1H-benzimidazole 4-carboxylic acid amide radical;
2-[2,2,5,5-tetramethyl-4-(3-trifluoromethyl-phenyl)-2,5-dihydro-1H-pyrrol-3-yl]-1H-benzimidazole 4-carboxylic acid amide;
2-[4-(1-oxyl-2,2,5,5-tetramethyl-2,5-dihydro-1H-pyrrol-3-yl)-phenyl]-1H-benzimidazole 4-carboxylic acid amide radical;
2-[4-(2,2,5,5-tetramethyl-2,5-dihydro-1H-pyrrol-3-yl)-phenyl]-1H-benzimidazole 4-carboxylic acid amide;
2-(1,2,2,5,5-pentamethyl-2,5-dihydro-1H-pyrrol-3-yl)-1H-benzimidazole 4-carboxylic acid amide;
2-(1-acetyl-2,2,5,5-tetramethyl-2,5-dihydro-1H-pyrrol-3-yl)-1H-benzimidazole 4-carboxylic acid amide;
2-(1-methoxy-2,2,5,5-tetramethyl-2,5-dihydro-1H-pyrrol-3-yl)-1H-benzimidazole 4-carboxylic acid amide;
2-[4-(dibenzofuran-4-yl)-1-oxyl-2,2,5,5-tetramethyl-2,5-dihydro-1H-pyrrol-3-yl)-phenyl]-1H-benzimidazole 4-carboxylic acid amide radical;
2-[4-(dibenzofuran-4-yl)-2,2,5,5-tetramethyl-2,5-dihydro-1H-pyrrol-3-yl)-phenyl]-1H-benzimidazole 4-carboxylic acid amide;
(1-hydroxy-2,2,6,6-tetramethyl-1,2,3,6-tetrahydro-pyridin-4-yl)-1H-benzimidazole 4-carboxylic acid amide;
2-(2,2,6,6-tetramethyl-1,2,3,6-tetrahydro-pyridin-4-yl)-1H-benzimidazole 4-carboxylic acid amide;
2-[4-(1-oxyl-2,2,5,5-tetramethyl-2,5-dihydro-1H-pyrrol-3-yl-methoxy)-phenyl]-1H-benzimidazole 4-carboxylic acid amide radical;
2-[4-(2,2,5,5-tetramethyl-2,5-dihydro-1H-pyrrol-3-yl-methoxy)-phenyl]-1H-benzimidazole 4-carboxylic acid amide;
2-[3-methoxy-4-(1-oxyl-2,2,5,5-tetramethyl-2,5-dihydro-1H-pyrrol-3-yl-methoxy)-phenyl]-1H-benzimidazole 4-carboxylic acid amide radical;
2-[3-methoxy-4-(2,2,5,5-tetramethyl-2,5-dihydro-1H-pyrrol-3-yl-methoxy)-phenyl]-1H-benzimidazole 4-carboxylic acid amide;
2-(5-oxyl-4,4,6,6-tetramethyl-4,6-dihydro-5H-thieno[2,3-c]pyrrol-2-yl)-1H-benzimidazole 4-carboxylic acid amide radical;
2-(4,4,6,6-tetramethyl-4,6-dihydro-5H-thieno[2,3-c]pyrrol-2-yl)-1H-benzimidazole 4-carboxylic acid amide;
2-(1-oxyl-2,2,5,5-tetramethyl-2,5-dihydro-1H-pyrrol-3-yl)-1H-benzimidazole 4-carboxylic acid isopropylamide radical;
2-(2,2,5,5-tetramethyl-2,5-dihydro-1H-pyrrol-3-yl)-1H-benzimidazole 4-carboxylic acid isopropylamide;
1-(2,2,5,5-tetramethyl-2,5-dihydro-1H-pyrrol-3-yl-methyl)1H-benzimidazole 4-carboxylic acid amide radical;
1-(2,2,6,6-tetramethyl-1,2,3,6-tetrahydro-pyridin-4-yl)-1H-benzimidazole 4-carboxylic acid amide;
2-(1-oxyl-2,2,5,5-tetramethyl-2,5-dihydro-1H-pyrrol-3-yl-methylsulphanyl)-1H-benzimidazole 4-carboxylic acid amide radical;
2-(2,2,5,5-tetramethyl-2,5-dihydro-1H-pyrrol-3-yl-methyl-sulphanyl)-1H-benzimidazole 4-carboxylic acid amide;
2-(1-oxyl-2,2,6,6-tetramethyl-1,2,3,6-tetrahydro-pyridin-4-yl-methylsulphanyl)-1H-benzimidazole 4-carboxylic acid amide; and
2-(2,2,6,6-tetramethyl-1,2,3,6-tetrahydro-pyridin-4-yl-methylsulphanyl)-1H-benzimidazole 4-carboxylic acid amide.

22. The pharmaceutical composition according to claim 19, wherein the salt is formed with inorganic or organic acids.

23. The pharmaceutical composition according to claim 19, wherein said salt is an oxalate, a hydrochloride, a hydrobromide, a sulphate, a phosphate, a phosphite, a borate, a lactate, an ascorbate, an acetate, a fumarate, a formiate, a tosylate, a tartarate, a maleate, a citrate, a gluconate, or a besylate.

24. The pharmaceutical composition according to claim 19, wherein the disease is selected from the group consisting of ischemia/reperfusion, inflammation, potentiation of cancer therapies, and combinations thereof.

25. The pharmaceutical composition according to claim 19, wherein said composition is formulated for a route of administration selected from the group consisting of oral, transdermal, parenteral, intramuscular, and intravenous.

26. The pharmaceutical composition according to claim 19, wherein said composition is formulated as a tablet, injection, solution, suppository, patch, or suspension.

27. A method for the preparation of a compound of the formula 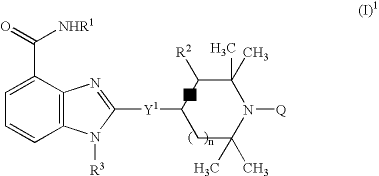
or a pharmaceutically acceptable or technically applicable salt thereof, wherein
R¹ represents hydrogen, C₍₁₋₄₎alkyl, or C₍₁₋₄₎alkoxy;
R² represents hydrogen, C₍₁₋₄₎alkyl, carboxyl, C₍₁₋₄₎alkoxycarbonyl, carboxamido, aryl, or hetero-aryl;
R³ represents hydrogen, C₍₁₋₄₎alkyl, aryl-methylene, or aryl;
Y¹ is a valency bond, a straight or branched C₍₁₋₄₎alkene, or a carbonyl-amino-C₍₁₋₄₎alkene;
n represents zero or the integer 1;
Q represents hydrogen, hydroxyl, or the oxygen radical (O.), or together with the N atom of the adjacent ring forms a +N=O (oxoimmonium) group;
Z represents a single or double bond; and
wherein any or all alkene groups may be spaced by an arylene group, comprising:
reacting a carboxamide of the formula 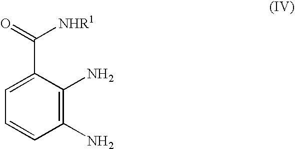
wherein R¹ has the meaning stated above, with a heterocyclic derivative of the formula 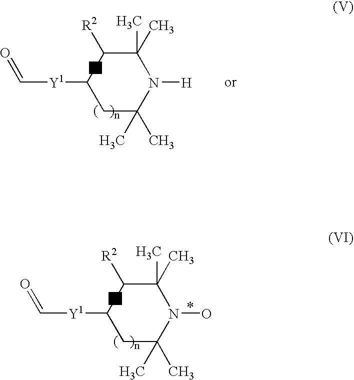
wherein R², Y¹, Z, and n have the meanings stated above.

28. The method of claim 27, wherein said salt is an oxalate, a hydrochloride, a hydrobromide, a sulphate, a phosphate, a phosphite, a borate, a lactate, an ascorbate, an acetate, a fumarate, a formiate, a tosylate, a tartarate, a maleate, a citrate, a gluconate, or a besylate.

29. A method for the preparation of a compound of the formula 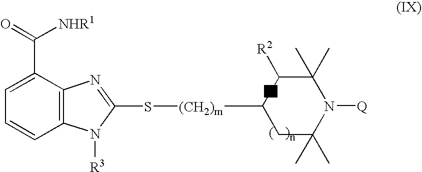
or a pharmaceutically acceptable or technically applicable salt thereof, wherein
R¹ represents hydrogen, C₍₁₋₄₎alkyl, or C₍₁₋₄₎alkoxy;
R² represents hydrogen, C₍₁₋₄₎alkyl, carboxyl, C₍₁₋₄₎alkoxycarbonyl, carboxamido, aryl, or hetero-aryl;
R³ represents hydrogen, C₍₁₋₄₎alkyl, aryl-methylene, or aryl;
n represents zero or the integer 1;
m represents the integer 1, 2, or 3;
Q represents hydrogen, hydroxyl, or the oxygen radical (O.), or together with the N atom of the adjacent ring forms a +N=O (oxoimmonium) group;
Z represents a single or double bond; and
wherein any or all alkene groups may be spaced by an arylene group, comprising:
reacting a compound of the formula 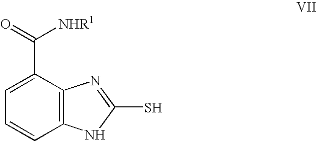
wherein R¹ has the meaning stated above, with an alkylating agent of the formula 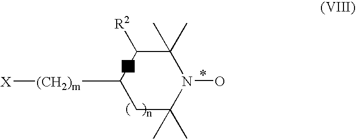
wherein R², Z, Q, n and m have the meanings stated above and X stands for a leaving group capable of reacting with the mercapto group to form a thioether, and optionally changing the substituents Q by way of oxidation and/or reduction to obtain the desired change in the substituents Q.

30. The method according to claim 29, wherein the compound of formula (VIII) is a correspondingly substituted alkyl-halogenide or alkyl-sulphonate and the reaction is carried out in the presence of a base.

31. The method according to claim 30, wherein the correspondingly substituted alkyl-halogenide or alkyl-sulphonate is a type selected from the group consisting of alkyl chloride, alkyl bromide, alkyl iodide, alkyl mesylate, alkel tosylate, and alkyl triflate.

32. The method of claim 29, wherein said salt is an oxalate, a hydrochloride, a hydrobromide, a sulphate, a phosphate, a phosphite, a borate, a lactate, an ascorbate, an acetate, a fumarate, a formiate, a tosylate, a tartarate, a maleate, a citrate, a gluconate, or a besylate.

33. A method for treating a disease that is based on PARP activation and/or are caused by Reactive Oxidative Species (ROS) and Reactive Nitrogen Species (RNS), comprising administering an effective dose of at least one compound of the formula 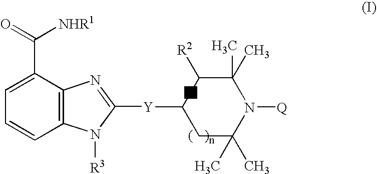
or a pharmaceutically acceptable or technically applicable salt thereof, wherein
R¹ represents hydrogen, C₍₁₋₄₎alkyl, or C₍₁₋₄₎alkoxy;
R² represents hydrogen, C₍₁₋₄₎alkyl, carboxyl, C₍₁₋₄₎alkoxycarbonyl, carboxamido, aryl, or hetero-aryl;
R³ represents hydrogen, C₍₁₋₄₎alkyl, aryl-methylene, or aryl;
Y is a valency bond, a straight or branched chain C₍₁₋₄₎alkene, a carbonyl-amino-C₍₁₋₄₎alkene, or a —S—(CH₂)ₘ— group;
n represents zero or the integer 1;
m represents the integer 1, 2, or 3;
Q represents hydrogen, hydroxyl, or the oxygen radical (O.), or together with the N atom of the adjacent ring forms a +N=O (oxoimmonium) group;
Z represents a single or double bond; and
wherein any or all alkene groups may be spaced by an arylene group,
in the form of a dosage form comprising said effective dose.

34. The method according to claim 33, wherein the disease is selected from the group consisting of ischemia/reperfusion, inflammation, unfavorable reaction in the course of radiotherapy or chemotherapy, and combinations thereof.
